(19) United States
(12) Patent Application Publication
(10) Pub. No.: US 2024/0226223 A1
(43) Pub. Date: Jul. 11, 2024

(54) COMPOSITION FOR PREVENTING, IMPROVING, OR TREATING MUSCLE LOSS COMPRISING GREEN TEA PEPTIDE COMPOSITION

(71) Applicant: AMOREPACIFIC CORPORATION, Seoul (KR)

(72) Inventors: Hyun Woo JEONG, Yongin-si (KR); Jinoh CHUNG, Yongin-si (KR); Wanki KIM, Yongin-si (KR); Jong Hwa ROH, Yongin-si (KR)

(73) Assignee: AMOREPACIFIC CORPORATION, Seoul (KR)

(21) Appl. No.: 18/536,404

(22) Filed: Dec. 12, 2023

(30) Foreign Application Priority Data
Jan. 6, 2023 (KR) 10-2023-0002399

(51) Int. Cl. A61K 36/82; A61K 38/16; A61P 21/00

(52) U.S. Cl. CPC A61K 36/82; A61K 38/168; A61P 21/00; A61K 2236/19; A61K 2236/331; A61K 2236/333

(57) ABSTRACT

The present disclosure relates to a composition for preventing, improving, or treating muscle loss comprising a green tea peptide composition. Specifically, the green tea peptide composition according to the present disclosure may exhibit an efficacy in preventing, improving, or treating muscle loss through the excellent effects of increasing protein synthesis in muscle cells and inhibiting protein degradation in the muscle cells, and thus may be applied in various health functional food compositions and pharmaceutical compositions.

### CROSS REFERENCE TO RELATED APPLICATION

The present application claims priority to Korean Patent Application No. 10-2023-0002399, filed on Jan. 6, 2023, the entire contents of which is incorporated herein for all purposes by this reference.

The instant application contains a Sequence Listing which has been submitted electronically in XML format and is hereby incorporated by reference in its entirety. Said XML copy, created on Oct. 20, 2023 is named 3549-136Seq.XML and is 8,192 bytes in size on disk.

### FIELD OF THE INVENTION

The present disclosure relates to a composition for preventing, improving, or treating muscle loss comprising a green tea peptide composition.

### BACKGROUND

The tea plant is one of 82 species classified in the genus Camellia and is currently cultivated in more than 50 countries, mainly in Asia, but also in Africa, South America, and Oceania. According to the processing method of tea leaves, the types of tea are broadly classified into unfermented tea, semi-fermented tea, fermented tea, and post-fermented tea. Among them, unfermented tea is made by inactivating the polyphenol oxidase contained in the tea plant by heat treatment, and contains more polyphenol types such as flavonol, flavanone, and flavonoid than other teas, exhibiting strong antioxidant power, and these substances account for approximately 30% of the dry weight of tea.

As the pharmacological mechanisms of various components in green tea have been gradually revealed, its value has been recognized by the general public, and in particular, the effects of antioxidant, anticancer, cholesterol-lowering, anti-aging, heavy metal detoxification, tooth decay prevention, and bad breath removal by polyphenols, the main components of green tea, have been proven, thus attracting wide attention.

Peptides are the most oxidation-stable substances in plants and are expected to have high skin efficacy due to their simple structure. In plants, the peptides act as a signal transmitting material and are known to be particularly involved in plant growth, differentiation, and response to external stimuli.

Meanwhile, athletic performance is an ability to be capable of quickly, strongly, long, and skillfully performing physical movements in daily life or sports. The athletic performance may be broadly classified as muscle fatigue recovery, endurance, and the like. The muscle fatigue is a state in which the physical activity performance is temporarily reduced after intense exercise or due to prolonged exercise, and is accompanied by a decrease in muscle contractility. The endurance is defined as a resistance to fatigue, which is a resistance to fatigue that occurs during maximal sustained exercise or intense exercise. A factor that contributes to fatigue during endurance exercise is the depletion of a substrate to provide energy to the muscles, and the fatigue occurs simultaneously with the depletion of glycogen stored in the muscles and liver. During high-intensity exercise, when the amount of oxygen supply is not enough to meet the oxygen consumption of the muscles, the concentration of lactic acid in the muscle tissues increases, in which case the lactic acid diffuses into the blood. Excessive accumulation of lactic acid from exercise leads to acidification in the body, which results in inhibition of gluconeogenesis, which is a source of energy for exercise in an anaerobic state.

The muscle not only serves as an organ of human athletic performance, but also affects the entire body, including bones, blood vessels, nerves, liver, heart, and pancreas. The bones are pulled and pushed by the muscles and maintain their density by that force, so when the muscles lose strength, the bones become weaker and more prone to developing osteoporosis. In addition, muscle loss prevents the development of new blood vessels and nerves due to the effects of various substances produced by the muscles, which may ultimately lead to a decrease in cognitive function.

The muscle loss, or muscle decrease, is a lifelong process that begins at around age 30 and progresses through the rest of the life span, during which the amount of muscle tissue, the number of muscle fibers, and the size of the muscle gradually decrease. The result of muscle loss is the gradual loss of muscle mass and muscle strength. The mild muscle loss increases stress on some joints, such as the knees, and may cause a person to be more vulnerable to arthritis or falls. In addition, fast-contracting muscle fibers are more affected by aging than slow-contracting muscle fibers. Therefore, as the aging progresses, it is difficult for the muscles to contract quickly, which leads to discomfort in daily life.

### SUMMARY OF THE INVENTION

The present disclosure aims to provide a composition including a green tea peptide that has a novel amino acid sequence isolated and purified by culturing a green tea protein and a specific plant-based lactic acid bacterium as an active ingredient, which exhibits an excellent muscle loss prevention, improvement or treatment effect.

To achieve the object described above, an embodiment of the present invention is directed to providing a composition for preventing, improving, or improving muscle loss, including a green tea peptide composition as an active ingredient.

The green tea peptide composition according to the present disclosure may exhibit an efficacy in preventing, improving, or treating muscle loss through the excellent effects of increasing protein synthesis in muscle cells and inhibiting protein degradation in the muscle cells, and thus may be applied in various health functional food compositions and pharmaceutical compositions.

### BRIEF DESCRIPTION OF THE DRAWINGS

FIG. 1 schematically illustrates a manufacturing process for a green tea peptide according to an embodiment of the present invention.

FIGS. 2A to 2C illustrate results of confirming a muscle loss-inducing marker expression inhibition effect by concentration of a green tea peptide composition (GTP) according to Example 1 (***P<0.001 vs. DEX, **P<0.01 vs. DEX, *P<0.05 vs. DEX).

FIGS. 3A to 3C illustrate results of confirming a protein synthesis acceleration effect in a muscle cell by concentration of the green tea peptide composition (GTP) according to Example 2 (***P<0.001 vs. DEX, **P<0.01 vs. DEX, *P<0.05 vs. DEX).

FIGS. 4A to 4C illustrate results of comparing the muscle loss inducing-marker expression inhibition effects of the green tea peptides according to a processing method according to Example 3 (***P<0.001 vs. DEX, **P<0.01 vs. DEX, *P<0.05 vs. DEX).

FIG. 5 illustrates a result of comparing the similarity between Lacticaseibacillus paracasei and Lactiplantibacillus plantarum.

### DETAILED DESCRIPTION OF THE INVENTION

Hereinafter, the present invention will be described in detail.

In the present specification, the term “green tea (tea; Camellia Sinensis)” refers to an evergreen broad-leaved shrub of the family Teaaceae, the tea from the dried leaves of which has been used in a variety of ways. In particular, the green tea is known to exhibit the effects of antioxidant, anticancer, and cardiovascular effects, reducing lipids in the blood and promoting blood circulation. The green tea includes one or more of those selected from the group consisting of tea plant leaves, flowers, stems, fruits, roots, stems, and heartwood of roots, and may preferably be leaves.

In the present specification, the term “active ingredient” means an ingredient that may exhibit the intended activity alone or in combination with a carrier that is not itself active.

In one aspect, the present invention may relate to a composition for preventing, improving, or treating muscle loss, including a green tea peptide composition as an active ingredient.

In another aspect, the present invention may relate to a method of preventing, improving, or treating muscle loss, including a step of administering an active amount of a green tea peptide composition to a subject in need thereof.

In still another aspect, the present invention may relate to a use of a green tea peptide composition for preparing a composition for preventing, improving, or treating muscle loss.

In an embodiment, the green tea peptide composition may include one or more species of green tea peptides including an amino acid sequence selected from the group consisting of SEQ ID NO: 1 to SEQ ID NO: 7. Specifically, the green tea peptide may include an amino acid sequence selected from the group consisting of AYKRRKGKFA (SEQ ID NO: 1), FFFFFFFFFFFFFFFYL (SEQ ID NO: 2), ISKIWNSEVPETEVKNEAESP (SEQ ID NO: 3), PFFCEKMMETN (SEQ ID NO: 4), RFLHERMAYYH (SEQ ID NO: 5), RNLNRLQRLLSMKQEYSPRNHLGSRWREY (SEQ ID NO: 6), and TTSSRKKEKPRRFWNNHEEVFLITTK (SEQ ID NO: 7).

In an embodiment, 60% (w/w) or more, 65% (w/w) or more, 70% (w/w) or more, 75% (w/w) or more, 80% (w/w) or more, 85% (w/w) or more, 90% (w/w) or more, or 95% (w/w) or more of the green tea peptide composition may be composed of one or more species of green tea peptides.

In an embodiment, the green tea peptide composition may be obtained by fermenting a green tea protein with a plant-based lactic acid bacteria.

In an embodiment, the fermentation may be performed at a condition of pH 5 to 8. Specifically, the fermentation may be performed at pH 5 or more, pH 5.2 or more, pH 5.4 or more, pH 5.6 or more, pH 5.8 or more, pH 6 or more, pH 6.2 or more, pH 6.4 or more, pH 6.6 or more, pH 6.8 or more, pH 7 or more, pH 7.2 or more, pH 7.4 or more, pH 7.6 or more or pH 7.8 or more. In addition, the fermentation may be performed at pH 8 or less, pH 7.8 or less, pH 7.6 or less, pH 7.4 or less, pH 7.2 or less, pH 7 or less, pH 6.8 or less, pH 6.6 or less, pH 6.4 or less, pH 6.2 or less, pH 6 or less, pH 5.8 or less, pH 5.6 or less, pH 5.4 or less or pH 5.2 or less. Preferably, the fermentation may be performed at pH 6.8.

In an embodiment, the fermentation may be performed at 25 to 45° C. Specifically, the fermentation may be performed at 25° C. or more, 27° C. or more, 29° C. or more, 31° C. or more, 33ºC or more, 35° C. or more, 37° C. or more, 39ºC or more, 41° C. or more or 43° C. or more. In addition, the fermentation may be performed at 45° C. or less, 43° C. or less, 41° C. or less, 39ºC or less, 37° C. or less, 35° C. or less, 33ºC or less, 31° C. or less, 29ºC or less or 27° C. or less. Preferably, the fermentation may be performed at 37° C.

In an embodiment, the fermentation may be performed for 24 to 72 hours. Specifically, the fermentation may be performed for 24 hours or more, 26 hours or more, 28 hours or more, 30 hours or more, 32 hours or more, 34 hours or more, 36 hours or more, 38 hours or more, 40 hours or more, 42 hours or more, 44 hours or more, 46 hours or more, 48 hours or more, 50 hours or more, 52 hours or more, 54 hours or more, 56 hours or more, 58 hours or more, 60 hours or more, 62 hours or more, 64 hours or more, 66 hours or more, 68 hours or more or 70 hours or more. In addition, the fermentation may be performed for 72 hours or less, 70 hours or less, 68 hours or less, 66 hours or less, 64 hours or less, 62 hours or less, 60 hours or less, 58 hours or less, 56 hours or less, 54 hours or less, 52 hours or less, 50 hours or less, 48 hours or less, 46 hours or less, 44 hours or less, 42 hours or less, 40 hours or less, 38 hours or less, 36 hours or less, 34 hours or less, 32 hours or less, 30 hours or less, 28 hours or less or 26 hours or less. Preferably, the fermentation may be performed for 48 hours.

In an embodiment, the plant-based lactic acid bacteria may be Lactiplantibacillus More specifically, the plant-based lactic acid bacterium may be plantarum. Lactiplantibacillus plantarum APsulloc 331261 (Korean Culture Center of Microorganisms, accession number KCCM11179P, accession date 20110328).

In an embodiment, the green tea protein may be obtained from a residue of a first extract that is extracted from green tea with anhydrous or hydrous C1-C6 low carbon alcohol.

In an embodiment, the concentration of alcohol in the hydrous C1-C6 low carbon alcohol may be 20 to 80% (v/v). Specifically, the concentration of alcohol in the hydrous C1-C6 low carbon alcohol may be 20% (v/v) or more, 22% (v/v) or more, 24% (v/v) or more, 26% (v/v) or more, 28% (v/v) or more, 30% (v/v) or more, 32% (v/v) or more, 34% (v/v) or more, 36% (v/v) or more, 38% (v/v) or more, 40% (v/v) or more, 42% (v/v) or more, 44% (v/v) or more, 46% (v/v) or more, 48% (v/v) or more, 50% (v/v) or more, 52% (v/v) or more, 54% (v/v) or more, 56% (v/v) or more, 58% (v/v) or more, 60% (v/v) or more, 62% (v/v) or more, 64% (v/v) or more, 66% (v/v) or more, 68% (v/v) or more, 70% (v/v) or more, 72% (v/v) or more, 74% (v/v) or more, 76% (v/v) or more or 78% (v/v) or more, also may be 80% (v/v) or less, 78% (v/v) or less, 76% (v/v) or less, 74% (v/v) or less, 72% (v/v) or less, 70% (v/v) or less, 68% (v/v) or less, 66% (v/v) or less, 64% (v/v) or less, 62% (v/v) or less, 60% (v/v) or less, 58% (v/v) or less, 56% (v/v) or less, 54% (v/v) or less, 52% (v/v) or less, 50% (v/v) or less, 48% (v/v) or less, 46% (v/v) or less, 44% (v/v) or less, 42% (v/v) or less, 40% (v/v) or less, 38% (v/v) or less, 36% (v/v) or less, 34% (v/v) or less, 32% (v/v) or less, 30% (v/v) or less, 28% (v/v) or less, 26% (v/v) or less, 24% (v/v) or less or 22% (v/v) or less.

In an embodiment, the hydrous C1-C6 low carbon alcohol may be a 20 to 80% (v/v) ethanol aqueous solution. Specifically, the hydrous C1-C6 low carbon alcohol may be a 20% (v/v) ethanol aqueous solution, a 25% (v/v) ethanol aqueous solution, a 30% (v/v) ethanol aqueous solution, a 35% (v/v) ethanol aqueous solution, a 40% (v/v) ethanol aqueous solution, a 41% (v/v) ethanol aqueous solution, a 42% (v/v) ethanol aqueous solution, a 43% (v/v) ethanol aqueous solution, a 44% (v/v) ethanol aqueous solution, a 45% (v/v) ethanol aqueous solution, a 46% (v/v) ethanol aqueous solution, a 47% (v/v) ethanol aqueous solution, a 48% (v/v) ethanol aqueous solution, a 49% (v/v) ethanol aqueous solution, a 50% (v/v) ethanol aqueous solution, a 51% (v/v) ethanol aqueous solution, a 52% (v/v) ethanol aqueous solution, a 53% (v/v) ethanol aqueous solution, a 54% (v/v) ethanol aqueous solution, a 55% (v/v) ethanol aqueous solution, a 56% (v/v) ethanol aqueous solution, a 57% (v/v) ethanol aqueous solution, a 58% (v/v) ethanol aqueous solution, a 59% (v/v) ethanol aqueous solution, a 60% (v/v) ethanol aqueous solution, a 65% (v/v) ethanol aqueous solution, a 70% (v/v) ethanol aqueous solution, a 75% (v/v) ethanol aqueous solution or a 80% (v/v) ethanol aqueous solution.

In an embodiment, the green tea protein may be obtained from a residue of a secondary extract which is hydrothermally extracted from the residue of the primary extract.

In an embodiment, the green tea protein may be obtained from the residue of the second extract through the processes of alkaline extraction, filtration and acid precipitation.

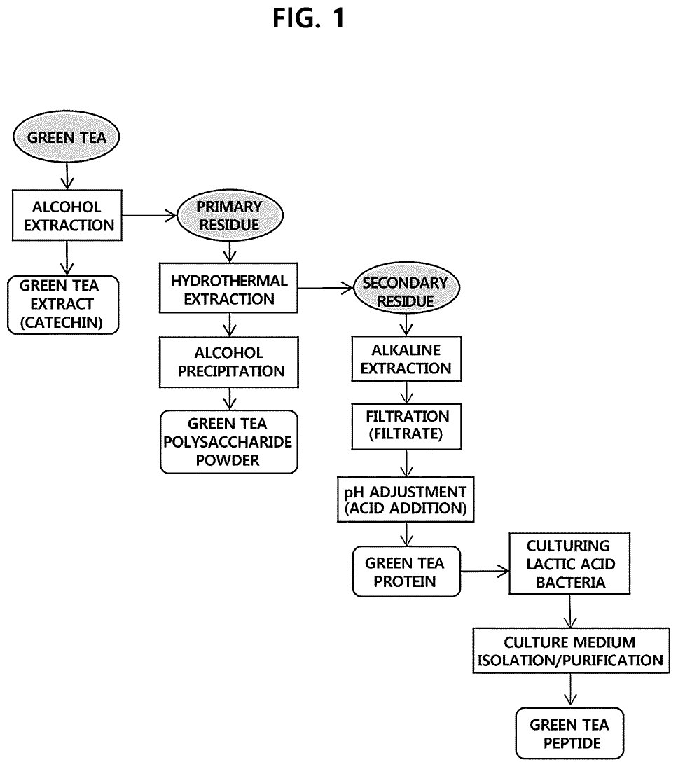

As illustrated in FIG. 1, the green tea peptide composition according to an embodiment of the present invention can be obtained by hydrothermal secondary extraction of the residue remaining after the primary extraction with green tea as a main alcohol, and by culturing the green tea protein obtained by alkaline extraction, filtration, and acid precipitation of the residue remaining after the secondary extraction with lactic acid bacterium.

In an embodiment, the green tea peptide composition may inhibit the expression of muscle loss inducing factor.

In an embodiment, the muscle loss inducing factor may be one or more selected from atrogin1, MuRF-1, and FoXO3.

In an embodiment, the green tea peptide composition may increase protein content in a muscle cell.

In an embodiment, the green tea peptide composition may be included in an amount of 1 to 50 wt % relative to a total weight of the composition for preventing, improving, or treating muscle loss. Specifically, the green tea peptide composition may be included in an amount of 1 wt % or more, 3 wt % or more, 5 wt % or more, 7 wt % or more, 10 wt % or more, 12 wt % or more, 14 wt % or more, 16 wt % or more, 18 wt % or more, 20 wt % or more, 22 wt % or more, 24 wt % or more, 26 wt % or more, 28 wt % or more, 30 wt % or more, 32 wt % or more, 34 wt % or more, 36 wt % or more, 38 wt % or more, 40 wt % or more, 42 wt % or more, 44 wt % or more, 46 wt % or more or 48 wt % or more relative to the total weight of the composition for preventing, improving, or improving muscle loss. In addition, the green tea peptide composition may be included in an amount of 50 wt % or less, 48 wt % or less, 46 wt % or less, 44 wt % or less, 42 wt % or less, 40 wt % or less, 38 wt % or less, 36 wt % or less, 34 wt % or less, 32 wt % or less, 30 wt % or less, 28 wt % or less, 26 wt % or less, 24 wt % or less, 22 wt % or less, 20 wt % or less, 18 wt % or less, 16 wt % or less, 14 wt % or less, 12 wt % or less, 10 wt % or less, 8 wt % or less, 7 wt % or less, 5 wt % or less or 3 wt % or less relative to the total weight of the composition for preventing, improving, or treating muscle loss.

In an embodiment, the green tea peptide composition may be administered in an amount of 1 to 400 mg/kg/day. Specifically, the green tea peptide composition may be administered in an amount of 1 mg/kg/day or more, 5 mg/kg/day or more, 10 mg/kg/day or more, 20 mg/kg/day or more, 30 mg/kg/day or more, 40 mg/kg/day or more, 50 mg/kg/day or more, 60 mg/kg/day or more, 70 mg/kg/day or more, 80 mg/kg/day or more, 90 mg/kg/day or more, 100 mg/kg/day or more, 150 mg/kg/day or more, 200 mg/kg/day or more, 250 mg/kg/day or more, 300 mg/kg/day or more or 350 mg/kg/day or more. In addition, the green tea peptide composition may be administered in an amount of 400 mg/kg/day or less, 350 mg/kg/day or less, 300 mg/kg/day or less, 250 mg/kg/day or less, 200 mg/kg/day or less, 150 mg/kg/day or less, 100 mg/kg/day or less, 90 mg/kg/day or less, 80 mg/kg/day or less, 70 mg/kg/day or less, 60 mg/kg/day or less, 50 mg/kg/day or less, 40 mg/kg/day or less, 30 mg/kg/day or less, 20 mg/kg/day or less, 10 mg/kg/day or less or 5 mg/kg/day or less.

In an embodiment, the composition for preventing, improving, or treating muscle loss may be a food composition. More specifically, the composition may be a health functional food composition for preventing, improving, or treating muscle loss.

The formulation of the food composition is not particularly limited, but may be formulated, for example, as tablets, granules, pills, powders, liquids such as beverages, caramels, gels, bars, tea bags, and the like. In addition to the active ingredient, the food composition of each formulation can be prepared by a person of ordinary skill in the art without difficulty, depending on the formulation or the purpose of use, with ingredients conventionally used in the art properly selected and combined, and synergistic effects may occur when applied simultaneously with other ingredients.

The composition may be administered in a variety of ways, including simple ingestion, drinking, injection administration, spray administration, or squeeze administration.

The food composition according to one aspect of the present invention may be, for example, a variety of food products, such as chewing gums, caramel products, candies, ice cream, confections, and the like; a beverage product, such as a soft drink, mineral water, or alcoholic beverage; or a health functional food product, including vitamins or minerals.

The food composition according to one aspect of the present invention may include food additives in addition to the active ingredient. The food additive may be generally understood as a material that is added to, mixed with, or infiltrated into food in the manufacturing, processing, or preservation of food, which will be consumed daily and for a long period of time with the food, and thus its safety should be ensured. The food additive code, which is based on the laws of each country that regulate the manufacture and distribution of food (in Korea, the “Food Hygiene Act”), stipulates that the food additives that are guaranteed to be safe are limited in terms of ingredients or functions. In the Korea Food Additives Code (Ministry of Food and Drug Safety's ┌Standards and Specifications for Food Additives┘), food additives are classified as chemical synthetics, natural additives, and mixed preparations in terms of ingredients, and these food additives are classified as sweeteners, flavoring agents, preservatives, emulsifiers, acidulants, and thickeners in terms of functions.

The sweetener is used to provide a moderate sweetness to the food product, and all the natural or synthetic sweetener may be used in the food composition according to one aspect of the present invention. Preferably, the natural sweetener is used, and examples of the natural sweeteners include corn syrup solids, honey, and sugar sweeteners such as sucrose, fructose, lactose, and maltose.

The flavoring agent is used to enhance taste or aroma, and all the natural and synthetic flavoring agents may be used. Preferably, the natural flavoring agent is used. When the natural flavoring agent is used, it may serve a nutritional purpose in addition to flavor. The natural flavoring agent may be one obtained from apples, lemons, tangerines, grapes, strawberries, peaches, and the like, or from green tea leaves, solomon's seal, bamboo leaves, cinnamon, chrysanthemum leaves, jasmine, and the like. In addition, the natural flavoring agent may be one obtained from ginseng (red ginseng), bamboo shoots, aloe vera, Ginkgo biloba, and the like. The natural flavoring agent may be a concentrate in the liquid phase or an extract in the solid phase. In some cases, the synthetic flavoring agent may be used, which may include ester, alcohol, aldehyde, terpene, and the like.

The preservatives may include calcium sorbate, sodium sorbate, potassium sorbate, calcium benzoate, sodium benzoate, potassium benzoate, and ethylenediaminetetraacetic acid (EDTA). In addition, the emulsifier may include acacia gum, carboxymethyl cellulose, xanthan gum, pectin, and the like, and the acidulant may include citric acid, malic acid, fumaric acid, adipic acid, gluconic acid, tartaric acid, ascorbic acid, acetic acid, phosphoric acid, and the like. The acidulant may be added to bring the food composition to a suitable acidity level for the purpose of inhibiting the growth of microorganisms in addition to the purpose of enhancing taste. The thickening agent may include a suspension enabler, a sedimentation agent, a gel-forming agent, a swelling agent, and the like.

The food composition according to one aspect of the present invention may include, in addition to the food additives described above, bioactive materials or minerals that are known in the art and whose stability as food additives is assured for the purpose of supplementing and enhancing functionality and nutrition.

The bioactive materials may include catechins, such as those found in green tea, vitamins such as vitamin B1, vitamin C, vitamin E, vitamin B12, tocopherols, dibenzoyl thiamine, etc., and the mineral may include calcium preparations such as calcium citrate, magnesium preparations such as magnesium stearate, iron preparations such as iron citrate, chromium chloride, potassium iodide, selenium, germanium, vanadium, zinc, and the like.

The food composition according to one aspect of the present invention may include the food additive as described above in a suitable amount to accomplish the purpose for which the food additive is added, depending on the type of product.

With respect to other food additives that may be included in the food composition according to one aspect of the present invention, the food code or food additive code of each country may be referenced.

In an embodiment, the composition may be a pharmaceutical composition for preventing, improving, or treating a disease related to muscle loss.

In an embodiment, muscle disease related to the muscle loss may be selected from the group consisting of sarcopenia, muscular atrophy, muscular dystrophy, and myasthenia gravis.

The sarcopenia refers to a disease in which normal muscle mass, muscle strength, and muscle function decrease due to poor nutrition, reduced exercise, and aging, while the muscular atrophy refers to a disease group of clinically and genetically diverse that exhibit symmetrical muscle weakness or loss due to reasons such as heredity.

The muscular dystrophy (MD) is a muscle disease that weakens athletic function and inhibits athletic performance, which has characteristics of progressive skeletal muscle weakness, muscle protein deficiency, and necrosis of muscle cells and tissues. The myasthenia gravis is a disease that causes abnormal weakness in the muscles and fatigue of the muscles. Without proper treatment, the myasthenia gravis may lead to sudden and severe muscle weakness and, in severe cases, respiratory muscle weakness, which may cause respiratory paralysis.

The pharmaceutical composition according to one aspect of the present invention may be prepared as an oral formulation or a parenteral formulation, depending on the route of administration, by conventional methods known in the art, including a pharmaceutically acceptable carrier in addition to the active ingredient. Here, the route of administration may be an arbitrary suitable route, including a topical route, an oral route, an intravenous route, an intramuscular route, and direct absorption through mucosal tissue, and a combination of two or more routes may be used. An example of the combination of two or more routes is a combination of two or more formulations of drugs by route of administration, for example, one drug is administered primarily by an intravenous route and the other drug is administered secondarily by a topical route.

The pharmaceutically acceptable carrier is known in the art, depending on the route of administration and formulation, and specifically the pharmacopoeia of each country, including the Korean Pharmacopoeia may be referenced.

When the pharmaceutical composition according to one aspect of the present invention is formulated in an oral formulation, the pharmaceutical composition may be prepared in the formulation of a powder, granule, tablet, pill, sugar coated tablet, capsule, liquid, gel, syrup, suspension, wafer, or the like according to methods known in the art in combination with a suitable carrier. In this case, examples of the suitable carriers may include sugars such as lactose, glucose, sucrose, dextrose, sorbitol, mannitol, and xylitol, starches such as corn starch, potato starch, and wheat starch, celluloses, such as cellulose, methylcellulose, ethylcellulose, sodium carboxymethylcellulose, and hydroxypropylmethylcellulose, polyvinylpyrrolidone, water, methylhydroxybenzoate, propylhydroxybenzoate, magnesium stearate, mineral oils, malt, gelatin, talc, polyols, vegetable oils, ethanol, and griseoil. For formulation, suitable binders, lubricants, disintegrants, colorants, diluents, and the like may be included as necessary. The suitable binders may include magnesium aluminum silicate, starch paste, gelatin, methylcellulose, sodium carboxymethylcellulose, polyvinylpyrrolidone, glucose, corn sweetener, sodium alginate, polyethylene glycol, wax, and the like, the lubricants may include sodium oleate, sodium stearate, magnesium stearate, sodium benzoate, sodium acetate, sodium chloride, silica, talcum, stearic acid, magnesium and calcium salts thereof, and polyethylene glycol, and the disintegrants may include methyl cellulose, agar, bentonite, xanthan gum, alginic acid, or sodium salts thereof. In addition, the diluents may include lactose, dextrose, sucrose, mannitol, sorbitol, cellulose, glycine, and the like.

When the pharmaceutical composition according to one aspect of the present invention is formulated in a parenteral formulation, the pharmaceutical composition may be formulated in the form of an injectable, transdermal, nasal inhalation, and suppository according to the method known in the art in combination with a suitable carrier. When the pharmaceutical composition is formulated as an injectable, an aqueous isotonic solution or suspension may be used as a suitable carrier, specifically phosphate buffered saline (PBS) containing triethanolamine, sterile water for injection, or an isotonic solution such as 5% dextrose. When formulated in a transdermal dosage form, the pharmaceutical composition may be formulated in the form of an ointment, cream, lotion, gel, topical solution, paste, liniment, aerosol, etc. When the pharmaceutical composition is formulated as a nasal inhalation, the pharmaceutical composition may be formulated as the form of an aerosol spray using a suitable propellant such as dichlorofluoromethane, trichlorofluoromethane, dichlorotetrafluoroethane, carbon dioxide, and the like, and when the pharmaceutical composition is formulated as a suppository, witepsol, tween 61, polyethylene glycol, cacao butter, laurin butter, polyoxyethylene sorbitan fatty acid esters, polyoxyethylene stearates, sorbitan fatty acid esters, and the like may be used as the carrier.

The amount of application or administration of the pharmaceutical composition according to one aspect of the present invention will depend on the age, sex, weight, pathologic condition and its severity of the subject to be administered, the route of administration, or the judgment of the prescriber. The determination of the amount of administered active ingredient based on these factors falls within the skill of a person of ordinary skill in the art.

The composition for preventing, improving, or treating muscle loss according to one aspect of the present invention may further include, in addition to the active ingredient, an arbitrary compound or natural extract already known in the art to be safe and to have the corresponding activity, in order to enhance or augment the muscle loss prevention, improvement or treatment effect or to improve the convenience of administration or ingestion through the addition of a similar activity, such as blood pressure regulating activity. This compounds or extracts include compounds or extracts listed in official documents such as the Korean Pharmacopoeia (in Korea, the “Korean Pharmacopoeia”), the Korean Health Functional Food Official Code (in Korea, the “Health Functional Food Standards and Specifications” notified by the Ministry of Food and Drug Safety), compounds or extracts that have been approved for products under the laws of each country governing the manufacture and sale of drugs (in Korea, the “Pharmaceutical Affairs Act”), and compounds or extracts whose functionality is certified under the laws of each country governing the manufacture and sale of health functional foods (in Korea, the “Health Functional Foods Act”).

For example, according to the Korean “Health Functional Foods Act”, the followings may correspond to these compounds or extracts: garcinia cambogia peel extract, conjugated linolenic acid (free fatty acid), conjugated linolenic acid (triglyceride), green tea extract, chitosan, Lactobacillus gasseri BNR17, L-carnitine tartrate, green maté extract, green coffee bean extract, sesame leaf extract, soybean germ extract, gynostemma leaf alcohol extract powder, lactoferrin (milk purified protein), lemon balm extract mixed powder, mate hydrothermal extract, complex extract of seaweed and others (Xanthigen), fermented vinegar pomegranate complex, boicha extract, mouse eye bean peptide complex, vegetable oil diglyceride, wild mango seed extract, oil containing medium chain fatty acids (MCFA), coleus forskohlii extract, chitooligosaccharides, finger root extract powder, complex extract of hibiscus and others, which are recognized for the functionality of ‘reducing body fat’, and L-glutamic acid-derived GABA-containing powder, katsuobushi oligopeptide, natto fungus culture powder, mouse eye bean peptide complex, salmon peptide, olive leaf extract, sardine peptide, casein hydrolysate, coenzyme Q10, grape seed enzymolysis extract powder, haetae oligopeptide, and the like, which are recognized for the functionality of ‘blood pressure control’, and DHA concentrated oil, globin hydrolysate, ovarian maltodextrin, bamboo leaf extract, vegetable oil diglycerides, sardine purified fish oil, purified squid oil, and the like, which are recognized for the functionality of ‘improving triglycerides in the blood’, and L-arabinose, nopal extract, cinnamon extract powder, guava leaf extract, nondigestible maltodextrin, freeze-dried silkworm powder, hemp alcohol extract, banaba leaf extract, morus alba extract, and the like, which are recognized for the functionality of ‘regulating blood sugar’, and fermentation-generated amino acid complex, hovenia dulcis fruit extract, Rhodiola rosea extract, and the like, which are recognized for the functionality of ‘improving fatigue’, and L-theanine, ashwagandha extract, whey protein hydrolysate, gynostemma leaf extract, and the like, which are recognized for the functionality of ‘anti-stress’.

In an example, the present invention may provide the following embodiments.

A first embodiment may provide a composition for preventing, improving, or treating muscle loss, including a green tea peptide composition as an active ingredient.

A second embodiment may provide the composition for preventing, improving, or treating muscle loss according to the first embodiment, in which the green tea peptide composition includes one or more species of green tea peptides including an amino acid sequence selected from the group consisting of SEQ ID NO: 1 to SEQ ID NO: 7.

A third embodiment may provide the composition for preventing, improving, or treating muscle loss according to one or more of the first and second embodiments, in which the green tea peptide composition is obtained by fermenting a green tea protein with plant-based lactic acid bacteria.

A fourth embodiment may provide the composition for preventing, improving, or treating muscle loss according to one or more of the first to third embodiments, in which the plant-based lactic acid bacteria is Lactiplantibacillus plantarum.

A fifth embodiment may provide the composition for preventing, improving, or treating muscle loss according to one or more of the first to fourth embodiments, in which the green tea protein is obtained from a residue of a primary extract that is extracted from green tea with anhydrous or hydrous C1-C6 low carbon alcohol.

A sixth embodiment may provide the composition for preventing, improving, or treating muscle loss according to one or more of the first to fifth embodiments, in which a concentration of an alcohol in the hydrous C1-C6 low carbon alcohol is 20 to 80% (v/v).

A seventh embodiment may provide the composition for preventing, improving, or treating muscle loss according to one or more of the first to sixth embodiments, in which the hydrous C1-C6 low carbon alcohol is an ethanol aqueous solution of 20 to 80% (v/v).

An eighth embodiment may provide the composition for preventing, improving, or treating muscle loss according to one or more of the first to seventh embodiments, in which the green tea protein is obtained from a residue of a secondary extract that is hydrothermally extracted from the residue of the primary extract.

A ninth embodiment may provide the composition for preventing, improving, or treating muscle loss according to one or more of the first to eighth embodiments, in which the green tea peptide composition inhibits an expression of a muscle loss inducing factor.

A tenth embodiment may provide the composition for preventing, improving, or treating muscle loss according to one or more of the first to ninth embodiments, in which the muscle loss inducing factor is one or more selected from atrogin1, MuRF-1, and FoXO3.

An eleventh embodiment may provide the composition for preventing, improving, or treating muscle loss according to one or more of the first to tenth embodiments, in which the green tea peptide composition increases protein content in a muscle cell.

A twelfth embodiment may provide the composition for preventing, improving, or treating muscle loss according to one or more of the first to eleventh embodiments, in which the green tea peptide composition is included in an amount of 1 to 50 wt % relative to a total weight of the composition.

A thirteenth embodiment may provide the composition for preventing, improving, or treating muscle loss according to one or more of the first to twelfth embodiments, in which the green tea peptide composition is administered in an amount of 1 to 400 mg/kg/day.

A fourteenth embodiment may provide the composition for preventing, improving, or treating muscle loss according to one or more of the first to thirteenth embodiments, in which the composition is a health functional food composition.

A fifteenth embodiment may provide the composition for preventing, improving, or treating muscle loss according to one or more of the first to fourteenth embodiments, in which the composition is a pharmaceutical composition for preventing, improving, or treating a disease related to muscle loss.

Hereinafter, the content of the present invention will be described in more detail through examples and test examples. However, these embodiments and examples are presented for the purpose of understanding the content of the present invention, and the scope of the present invention is not limited to these embodiments and examples, and modifications, substitutions, and insertions known in the art can be made, which are also included in the scope of the present invention.

### [Example 1] Preparation of Green Tea Peptide Composition

The green tea of 50 kg (Camelia sinensis, Agricultural Corporation Osulloc Farm) was added to a 1-ton extraction tank, ethanol of 50% (v/v) was added at 15 times, and then extracted (primary extraction) at 70° ° C. for 2 hours and filtered to remove catechins and obtain a residue of primary extract of the green tea. Purified water was added to the residual solids of the primary extract of the green tea at a ratio of 15 times, which was then extracted (secondary extraction) at 90° ° C. for 3 hours and filtered to remove water-soluble polysaccharides, etc. to obtain the residue of the secondary extract of the green tea. A 2% (w/w) aqueous solution of NaOH (98%, Youngjin Co., Ltd.) was added to the residual solids of the secondary extract of the obtained green tea at a ratio of 10 times, which was extracted (alkaline extraction) at 70° C. for three hours and filtered to obtain the filtrate. The obtained filtrate was cooled to room temperature and 35% (w/w) hydrochloric acid (Daejung Chemicals) was added to adjust the pH to 3.5 to 4.5 or less. The supernatant was removed and the precipitate was washed three to seven times with purified water, and then the precipitate was spray dried using an Ohkawara OC-16 spray dryer (inlet 220° C., outlet 90)° ° C. to obtain the green tea protein with a crude protein content of 50% (w/w) or more. Lactiplantibacillus plantarum APsulloc 331261 culture medium (containing purified water, vitamin solution, amino acid solution, and mineral solution) containing the green tea protein of 1% (w/w) was added to an anaerobic fermenter and incubated at pH 6.8 and 37° ° C. for 48 hours, and then the culture medium was centrifuged (Labogene 1580R (Serial No. KLG4226180220023) at 4° C., 10,000 g for 20 minutes to obtain a supernatant. The obtained supernatant was concentrated in a dry oven at 70° C., and the concentrate was centrifuged (Hitachi centrifuge CS150NX) at 4° C., 100,000 g for one hour to obtain a supernatant. The obtained supernatant was subjected to membrane filtration (pore size 0.22 μm) to obtain a filtrate, and the obtained filtrate was freeze-dried to obtain the green tea peptide composition (green tea peptide; GTP) (green tea peptide content 14% (w/w)).

Meanwhile, the sequence of the green tea peptide contained in the green tea peptide composition was analyzed in the following steps:
1) Centrifuging the Lactiplantibacillus plantarum APsulloc 331261 culture medium containing the green tea protein to separate the supernatant, and obtaining only the peptide fractionated in the low molecular weight peptide fraction by membrane filtration and size exclusion chromatography;
2) freeze-drying and de-salting the peptide obtained from 1), then dissolving the peptide in 0.1% (w/w) formic acid and analyzing the peptide by LC-MS/MS,

In this case, LC-MS/MS analysis was performed on 3 μg of sample (based on protein quantification), and the equipment and analysis conditions used were as follows;

TABLE 1

| | |
|---|---|
| LC | UltiMate 3000 RSLC nano system |
| equipment | |
| MS | Q-Exactive Orbitrap HF-X mass spectrometer (Thermo |
| equipment | Fisher Scientific) |
| Column | (trap) Internal diameter: 75 μm × 2 cm, packed with Acclaim |
| | PepMap 100 C18, 3 um(analytical) Internal diameter: 75 μm × |
| | 50 cm, packed with PepMap RSLC C18, 2 μm |
| Mobile | (solvent A) 0.1% (w/w) formic acid (FA) in water(solvent B) |
| phase | 0.1% (w/w) FA in acetonitrile |
| Mode | Positive ion mode |
| Collision | 27% |
| energy | |

3) Searching for peptide sequences using the green tea (camelia sinensis [UniProt Proteome ID: UP000327468]) and lactic acid bacterium (L. plantarum DSM 20174 [NCBI accession: GCA_014131735.1], L. plantarum APsulloc 331261) proteome sequence database for the spectra files obtained through LC-MS/MS analysis in 2). In this case, the analysis conditions used were as follows.

TABLE 2

| | |
|---|---|
| Search engine | MS-GF+ |
| Modifications | (fixed) None(variable) oxidation (+15.99) of |
| | methionine & acetylation (+42.01) of the peptide |
| | N-terminal |
| m/z tolerance | Precursor: ±10 ppmFragment: ±20 ppm |
| False | 1% |
| discovery rate cut-off | |

The green tea peptide sequences according to an embodiment of the present invention analyzed by the above steps is as follows.

TABLE 3

| | |
|---|---|
| AYKRRKGKFA | SEQ ID NO: 1 |
| FFFFFFFFFFFFFFFYL | SEQ ID NO: 2 |
| ISKIWNSEVPETEVKNEAESP | SEQ ID NO: 3 |
| PFFCEKMMETN | SEQ ID NO: 4 |
| RFLHERMAYYH | SEQ ID NO: 5 |
| RNLNRLQRLLSMKQEYSPRNHLGSRWREY | SEQ ID NO: 6 |
| TTSSRKKEKPRRFWNNHEEVFLITTK | SEQ ID NO: 7 |

### [Experimental Example 1] Confirmation of Muscle Loss Inducing-Marker Expression Inhibition Effect by Treatment by Concentration of Green Tea Peptide Composition

Muscle loss occurs when the consumption (degradation) of the protein that consists of the muscle fibers, etc. is greater than the protein synthesis (protein synthesis <<protein degradation). The protein degradation is mainly carried out by ubiquitin, and atrogin1, MuRF-1, etc. act as key factors in muscle loss, and FoXO3a or the like are known as transcription factors that regulate these ubiquitin-degrading proteins.

When the muscle cell is treated with dexamethasone (DEX), the expression of atrogin1 and MuRF-1 increases along with FoXO3. An experiment was carried out to confirm whether the green tea peptide composition according to one aspect of the present invention may inhibit the expression of the muscle loss inducing-marker as follows.

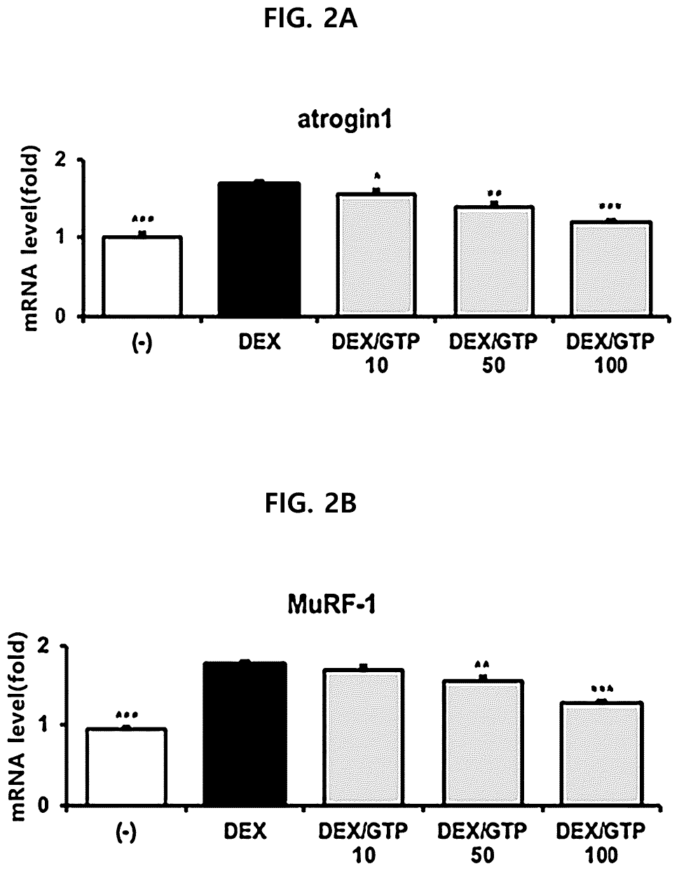

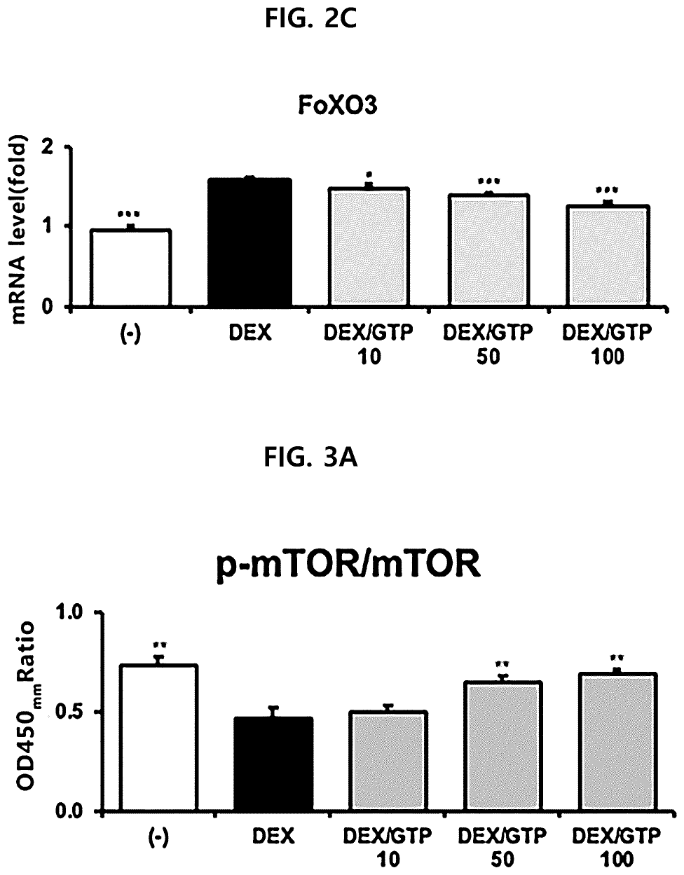

The C2C12 mouse-derived muscle cell line (ATCC) was cultured to 100% confluency in DMEM (Sigma Aldrich)+10% bovine calf serum (Gibco) medium and then induced to differentiate into muscle cells in DMEM+2% horse serum (Gibco) medium for 7 days. The differentiated muscle cells were treated with DEX (10 mM) and the green tea peptide composition (GTP) of Example 1 above at concentrations of 10, 50, and 100 mg/ml for 12 hours and the cells were collected. Then, TaKaRa MiniBEST Universal RNA Extraction Kit (Takara Bio) and RevertAid 1st-strand cDNA Synthesis Kit (Thermo Fisher Scientific) were sequentially used to complete RNA extraction and cDNA synthesis. The synthesized cDNA was used as a template for gene expression observation using CFX96 thermocycler (Bio-Rad) equipment. The results are illustrated in FIGS. 2A to 2C.

As illustrated in FIGS. 2A to 2C, the expression of atrogin1, MuRF-1 and FoXO3, which are the muscle loss inducing-markers, increased simultaneously upon the DEX treatment, in which case the expression of the muscle loss inducing-markers gradually and significantly decreased with the treatment concentration of the green tea peptide composition. It can be seen from this that the green tea peptide composition according to an embodiment of the present invention will effectively inhibit the induction of muscle loss.

### [Experimental Example 2] Confirmation of Increase in Protein Synthesis in Muscle Cell by Treatment by Concentration of Green Tea Peptide Composition

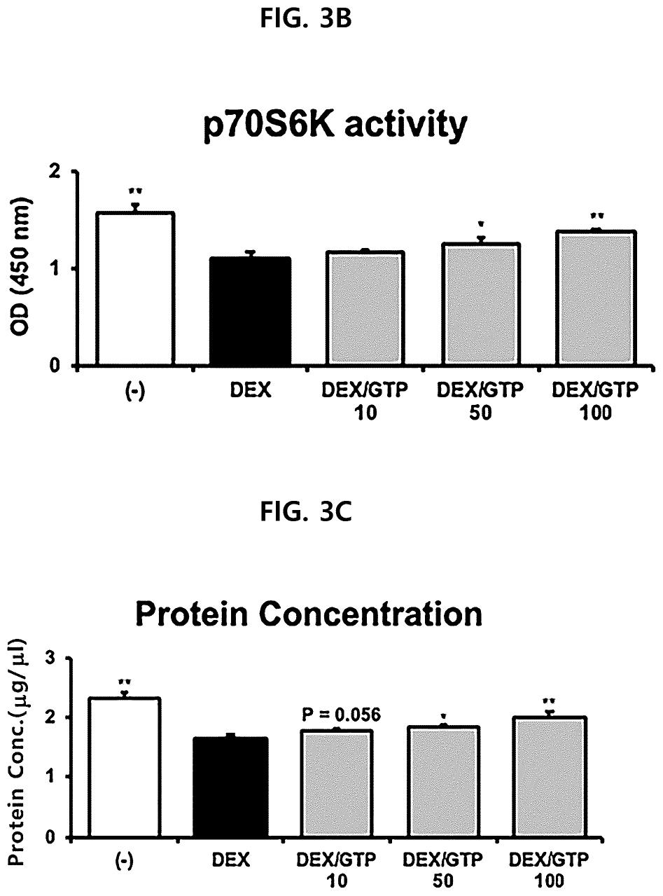

When hormones such as insulin activate mTOR through a signal transduction system (p-mTOR), p70 S6 kinase is activated by the mTOR (p-p70 S6 kinase), which regulates such that various roles such as cell survival, gene transcription, and protein synthesis are performed. Therefore, the activity of the mTOR and p70 S6 kinase, which are protein synthesis signals, was confirmed to confirm whether the green tea peptide composition according to one aspect of the present invention is capable of promoting protein synthesis in the muscle cells. As in Example 1 above, the differentiated muscle cells were treated with DEX (10 mM) and the green tea peptide composition (GTP) of Example 1 above at concentrations of 10, 50, and 100 mg/ml for 12 hours and the cells were collected. The proteins were extracted from the collected cells using a RIPA lysis buffer (Thermo Fisher Scientific), and then a certain amount of proteins were subjected to p-mTOR/total mTOR ELISA kit (Ray Biotech) and p70 S6 kianse (pT389) ELISA kit (Abcam) to measure the activity of each protein, and the results are illustrated in FIGS. 3A and 3B, respectively.

As illustrated in FIGS. 3A and 3B, it was confirmed that the green tea peptide composition according to an embodiment of the present invention activates the protein synthesis signal transduction system (mTOR and p70 S6 kinase).

Next, the differentiated muscle cells were treated with the DEX (10 mM) and the green tea peptide composition of Example 1 at concentrations of 10, 50, and 100 mg/ml for 48 hours to confirm whether the mTOR activation by the green tea peptide composition according to one aspect of the present invention actually induced protein synthesis in the muscle cells. The proteins were extracted from the collected cells using the RIPA buffer, and the protein content in the cell was subsequently quantified using a Bradford Assay Buffer (Invitrogen). The results are illustrated in FIG. 3C.

As illustrated in FIG. 3C, it was confirmed that the green tea peptide composition according to an embodiment of the present invention significantly increased the protein content in the muscle cell by activating the protein synthesis signal in the muscle cell, as predicted from the results of FIGS. 3A and 3B above.

### [Experimental Example 3] Confirmation of Muscle Loss Inducing-Marker Expression Inhibition Effect of Green Tea Peptide Composition According to Different Preparation Methods

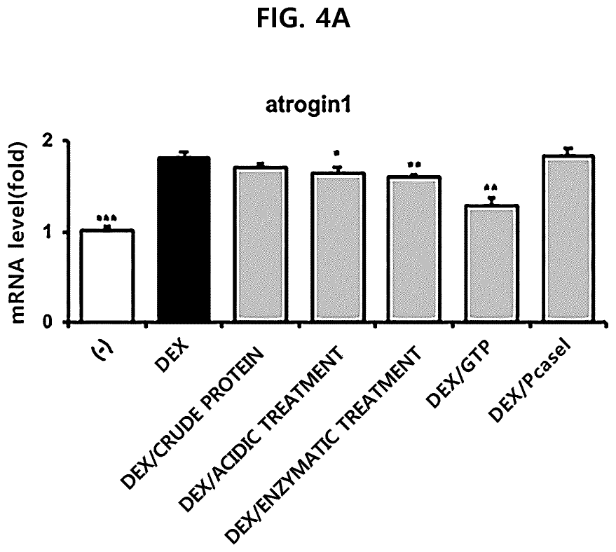

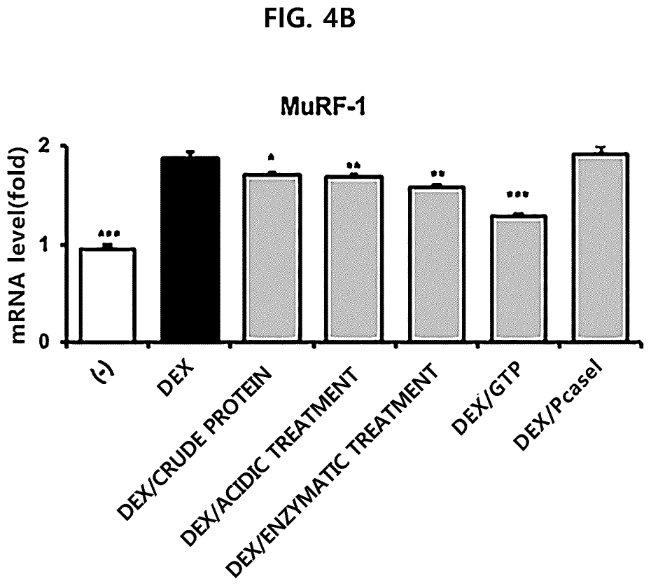

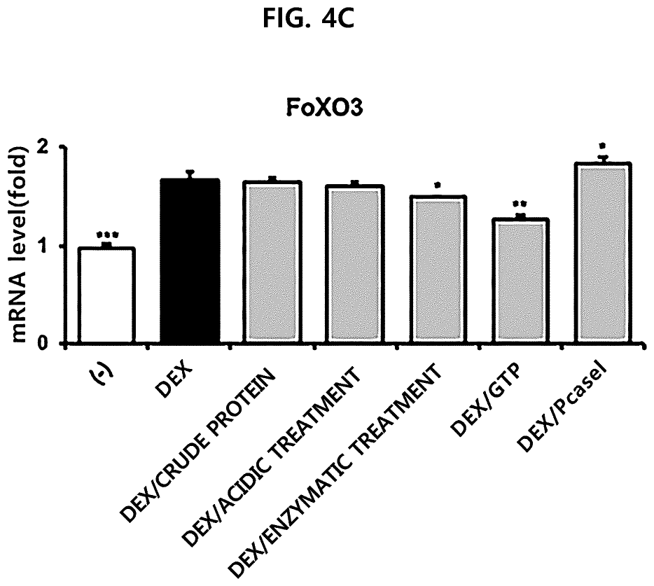

To compare the muscle loss inhibition efficacy of a green tea peptide according to a method of preparation, i) green tea crude protein obtained during the process of preparing the green tea peptide composition of Example 1 above, ii) a supernatant (green tea protein acid-treated fraction) obtained by preparing the green tea protein obtained during the process of preparing the green tea peptide composition of Example 1 above at a concentration of 1:40 (w/v) using purified water, followed by the addition of 35% (w/w) hydrochloric acid to adjust to pH 5, and then hydrolyzing the mixture at 37° C. for 6 hours, followed by centrifugation at 4° C., 10,000 g (Labogene 1580R (Serial No. KLG4226180220023), iii) a supernatant (a green tea proteolytic enzyme-treated fraction) obtained by preparing the green tea protein obtained during the process of preparing the green tea peptide composition of Example 1 above at a concentration of 1:40 (w/v) using 0.1 M sodium phosphate buffer, pH 8, followed by the addition of the proteolytic enzyme bromelain at 1% (w/v) relative to the green tea protein, and then hydrolyzing the mixture at 45° C., pH 6.2 for 24 hours, and heating the mixture at 90° C. for 30 minutes, followed by centrifugation at 4° C., 10,000 g (Labogene 1580R (Serial No. KLG4226180220023)), and iv) a peptide composition prepared in the same manner as in Example 1 above, except that Lacticaseibacillus paracasei (KCTC 3510 (ATCC Strain No.: ATCC 25302), Korean Collection for Type Cultures (KCTC)), which is an animal-based lactic acid bacterium, was used instead of Lactiplantibacillus plantarum APsulloc 331261, were treated together to compare the muscle loss prevention efficacy. The specific experimental method was performed as in Experimental example 1 above, except that the green tea crude protein, the green tea protein acid-treated fraction, the green tea proteolytic enzyme (bromelain)-treated fraction, and the peptide composition fermented by Lacticaseibacillus paracasei (L. paracasei), which is an animal-based lactic acid bacterium, and the green tea peptide composition according to Example 1 were each treated with the DEX (10 μM) at a concentration of 100 μg/ml for 12 hours. The results are illustrated in FIGS. 4A and 4B.

From the results of FIGS. 4A to 4C, it was confirmed that the green tea crude protein, degradation peptide, enzymatic degradation peptide, and animal-based lactic acid bacteria fermented peptide compositions exhibited no or negligible expression inhibition efficacy against atrogin 1, MuRF-1, and FoXO3, which are the muscle loss inducing-markers, while the green tea peptide composition treatment group according to an embodiment of the present invention exhibited a significantly excellent muscle loss inducing-marker expression inhibition activity.

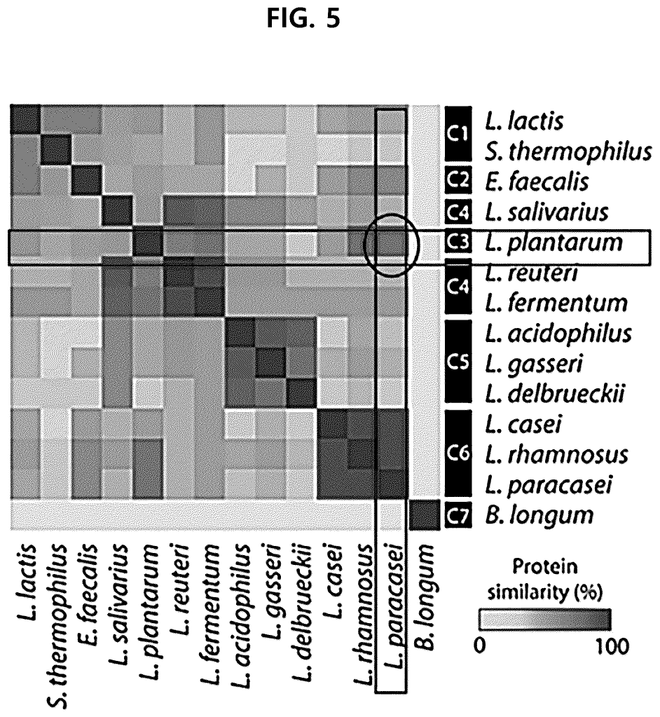

Meanwhile, FIG. 5 illustrates a result of analyzing the similarity between Lactiplantibacillus plantarum and Lacticaseibacillus paracasei. Similarity analysis was calculated by downloading the genome information (genbank) for each strain from NCBI (https://www.ncbi.nlm.nih.gov/genbank/), retaining only the annotated conserved protein genetic information, discarding the rest thereof, followed by calculation as a percentage of the total number. In this case, the R program was used to calculate the percentage of conserved proteins (POCP) (code source: https://github.com/hoelzer/pocp.git), and the analysis conditions were as follows:
E-value=1 Xe⁻⁵
Sequence identity=0.4
Alignment length=0.5.

Among these, two genomes were categorized into the same cluster when they exhibited 50% or more of similarity, and Lactiplantibacillus plantarum and Lacticaseibacillus paracasei were found to be very similar with 46.84% of similarity. As described above, it was confirmed that the green tea peptide composition (PCasei) obtained by fermentation with Lacticaseibacillus paracasei, which is an animal-based lactic acid bacterium very similar to the Lactiplantibacillus plantarum used in Example 1, did not exhibit the muscle loss inducing-marker gene inhibition efficacy, i.e., muscle loss prevention efficacy. Therefore, it can be seen that the muscle loss prevention efficacy of the green tea peptide composition according to an embodiment of the present invention is obtained through fermentation using Lactiplantibacillus plantarum, which is a plant-based lactic acid bacterium.

### [Accession Number]

Name of depository authority: Korea Culture Center of Microorganisms (overseas)

Accession Number: KCCM11179P

Accession Date: 2011328

What is claimed is:

1. A method of preventing, improving, or treating muscle loss, the method comprising:
administering an active amount of a green tea peptide composition to a subject in need thereof.

2. The method of claim 1, wherein the green tea peptide composition includes one or more species of green tea peptides including an amino acid sequence selected from the group consisting of SEQ ID NO: 1 to SEQ ID NO: 7.

3. The method of claim 1, wherein the green tea peptide composition is obtained by fermenting a green tea protein with a plant-based lactic acid bacterium.

4. The method of claim 3, wherein the plant-based lactic acid bacterium is Lactiplantibacillus plantarum.

5. The method of claim 3, wherein the green tea protein is obtained from a residue of a primary extract that is extracted from green tea with anhydrous or hydrous C1-C6 low carbon alcohol.

6. The method of claim 5, wherein a concentration of alcohol in the hydrous C1-C6 low carbon alcohol is 20 to 80% (v/v).

7. The method of claim 6, wherein the hydrous C1-C6 low carbon alcohol is a 20 to 80% (v/v) ethanol aqueous solution.

8. The method of claim 5, wherein the green tea protein is obtained from a residue of a secondary extract which is hydrothermally extracted from the residue of the primary extract.

9. The method of claim 1, wherein the green tea peptide composition inhibits an expression of a muscle loss inducing factor.

10. The method of claim 9, wherein the muscle loss inducing factor is one or more selected from atrogin1, MuRF-1, and FoXO3.

11. The method of claim 1, wherein the green tea peptide composition increases protein content in a muscle cell.

12. The method of claim 1, wherein the green tea peptide composition is formulated into a composition for preventing, improving, or treating muscle loss, and wherein the green tea peptide composition is included in an amount of 1 to 50 wt % by weight of a total weight of the composition for preventing, improving, or treating muscle loss.

13. The method of claim 1, wherein the green tea peptide composition is administered in an amount of 1 to 400 mg/kg/day.

14. The method of claim 1, wherein the green tea peptide composition is formulated into a health functional food composition for preventing, improving, or treating muscle loss.

15. The method of claim 1, wherein the green tea peptide composition is formulated into a pharmaceutical composition for preventing or treating a disease related to muscle loss.


SEQUENCE LISTING (7 sequences)

SEQ ID NO: 1 - protein, 10 aa, unidentified
AYKRRKGKFA

SEQ ID NO: 2 - protein, 17 aa, unidentified
FFFFFFFFFFFFFFFYL

SEQ ID NO: 3 - protein, 21 aa, unidentified
ISKIWNSEVPETEVKNEAESP

SEQ ID NO: 4 - protein, 11 aa, unidentified
PFFCEKMMETN

SEQ ID NO: 5 - protein, 11 aa, unidentified
RFLHERMAYYH

SEQ ID NO: 6 - protein, 29 aa, unidentified
RNLNRLQRLLSMKQEYSPRNHLGSRWREY

SEQ ID NO: 7 - protein, 26 aa, unidentified
TTSSRKKEKPRRFWNNHEEVFLITTK